(19) United States
(12) Patent Application Publication
(10) Pub. No.: US 2011/0009486 A1
(43) Pub. Date: Jan. 13, 2011

(54) METHOD FOR TREATING PRURITUS BY ADMINISTERING FULLERENES

(75) Inventors: Christopher Kepley, Ringgold, VA (US); Robert P. Lenk, Danville, VA (US); Stephen R. Wilson, Danville, VA (US); Zhiguo Zhou, Winston-Salen, NC (US)

Correspondence Address: BUCHANAN, INGERSOLL & ROONEY PC, POST OFFICE BOX 1404, ALEXANDRIA, VA, 22313-1404, US

(73) Assignee: Luna Innovations Incorporated, Roanoke, VA (US)

(21) Appl. No.: 12/921,072

(22) PCT Filed: Mar. 3, 2009
(86) PCT No.: PCT/US09/01332

§ 371 Date: Sep. 3, 2010

Related U.S. Application Data
(60) Provisional application No. 61/033,324, filed on Mar. 3, 2008.

(51) Int. Cl. A61K 31/216; A61P 17/04; C07C 69/76

(52) U.S. Cl. 514/510; 560/76

(57) ABSTRACT

Disclosed herein are methods of treating pruritus in a subject in need thereof. A method of treating pruritus comprises administering a therapeutically effective amount of fullerenes to a subject in need thereof.

### BACKGROUND

Various embodiments described herein relate to methods of treating pruritus (in Latin “prurire” means to itch).

Pruritus is a common and distressing symptom in a variety of diseases. Pruritus is a common dermatological symptom which can give rise to considerable distress, in both humans and animals. Pruritus is often associated with inflammatory skin disease which can commonly be caused by hypersensitivity reactions, such as reaction to insect bites e.g., flea bites, or to environmental allergens such as house dust mite or pollen; or by bacterial and fungal infections of the skin or ectoparasite infections. Pruritus typically occurs in peripheral diseases such as allergic conjunctivitis, allergic rhinitis, hemorrhoids, and dermatoses of fungal, allergic and non-allergic origin. Itching can also be a major symptom of certain systemic diseases such as, Hodgkin's disease, chronic renal failure, polycythema vera, hyperthyroidism and cholestasis See, e.g., Herndon, J. H. Jr., Int. J. Derm. 14, 465-484 (1975); Winkelmann, R. K., Med. Clins. N. Am. 66, 1119-1133 (1982).

Previous treatments for pruritus include the use of corticosteroids and antihistamines, however both have undesired side effects. Other therapies for pruritus include the use of essential fatty acid dietary supplements which are slow to act and offer only limited efficacy against allergic dermatitis. A variety of emollients such as soft paraffin, glycerine and lanolin are also employed but with limited success and there is a continuing need for an effective remedy.

It is known that certain opioids can precipitate asthma attacks in sensitive patients, and that morphine and related opioids are to be avoided during asthma attacks due to inherent respiratory depressant activity. It is also known that morphine may cause pruritus in certain individuals, which may be related to the degranulation of mast cells. Degranulation of mast cells is believed to play a central role in causing allergic responses.

Bernstein and Swift, Arch. Dermatol., 155:1366 (November 1979) have shown that subcutaneous administration of the narcotic antagonist naloxone blocked pruritus caused by primary biliary cirrhosis. The same article shows that administering an enkephalin analog with opioid-like activity precipitated an asthma attack in one patient. Bernstein U.S. Pat. No. 4,181,726 discloses treatment of itching associated with Hodgkin's disease, mycosis fungoides, intractable pruritus and the like by injection of naloxone. Parenteral administration of naloxone for treating antigen-induced pruritus, asthma, urticaria and angioedema is similarly disclosed by Smitz et al., Ann. Intern. Med., 97(5): 788-90 (1982).

It is well recognized in the field of opiate agonism and antagonism that there are multiple opiate receptor sub-species, each receptor having its own particular affinity for narcotics and antagonists thereof. The presence of multiple receptors for opiates and antagonists thereof creates a general unpredictability in the structure-activity relationship for narcotics and narcotic antagonists, as described in Goodman and Gilman's The Pharmacological Basis of Therapeutics, 6th ed.

Intravenous administration of opiate analgesics, such as morphine and hydromorphone has been associated with pruritus, urticaria, other skin rashes, and wheal and flare over the vein being injected. These itch and itch-related reactions are believed to be due to a histamine-releasing property of these opiates, via mast cell degranulation. These opiates are thought to act upon the mu subtype of opiate receptor, but the possibility of interactions at the other principal opiate receptor subtypes (delta and kappa) cannot be excluded since these and other pruritogenic analgesics are not pure mu agonists. The cellular loci of the receptor type(s) mediating the itching effect is not known, although the mast cell is a possible candidate since opiates cause histamine release from these cells. However, some investigators have suggested that the frequent inability of antihistamines to block morphine-induced itching suggests a non-histaminergic mediation of opiate-induced itching-mechanism which could involve central opiate receptors. Although i.v. morphine only occasionally results in general itching (in about 1% of patients), pruritus is more prevalent in opiate analgesia with epidural (8.5%) or intraspinal (45.8%) administration. See, e.g.,Bernstein et al., “Antipruritic Effect of an Opiate Antagonist, Naloxone Hydrochloride, The Journal of Investigative Dermatology,” 78:82-83 (1982); Ballantyne et al., “Itching after epidural and spinal opiates,” Pain, 33: 149-160 (1988).

To date, treatment with opiates has not only proven useless in the treatment of pruritus, but appears to exacerbate pruritus in mammals. The consistent findings from human studies indicate that whether by central or peripheral mechanisms, opiates appear to promote rather than prevent itching, and that opiate antagonists have antipruritic activity.

Human clinical studies have generally shown that opiates cause itching and there is evidence that these effects can be reproduced in animal models, where itching sensations per se cannot be reported, but scratching behavior can be observed. See, e.g., Thomas et al., “Microinjection of morphine into the rat medullary dorsal horn produces a dose-dependent increase in facial-scratching,” Brain Research, 695: 267-270 (1996); Thomas et al., “Effects of central administration of opioids on facial scratching in monkeys,” Brain Res., 585: 315-317 (1992); Thomas et al., “The medullary dorsal horn: A site of action of opioids in producing facial scratching in monkeys,” Anesthesiology, 79: 548-554 (1993).

Fullerenes are a family of carbon allotropes that comprise closed cages of generally 20 to 200 carbon atoms and may also include chemical moieties attached to the exterior or incorporated within the cage. Fullerenes can be in the form of a hollow sphere, ellipsoid, or tube. The most common fullerene to date is the C₆₀ Buckminsterfullerene (IUPAC name (C₆₀-Ih)[5,6]fullerene). Another fairly common buckminsterfullerene is C₇₀, but fullerenes with 72, 76, 84 and even up to 200 carbon atoms can be obtained. Fullerenes can contain 500 or more carbon atoms.

Structural variations include nonclosed-cage structures, heterofullerenes, derivatives formed by substitution of hydrofullerenes, the fusion of organic rings or ring systems to the fullerene cage, chiral fullerenes, buckyball clusters, nanotubes, megatubes, polymers, nano “onions,” linked “ball-and-chain” dimers, and fullerene rings. See, e.g., Miessler and Tarr, Inorg. Chem. 3, Pearson Education International. ISBN 0-13-120198-0 (2004); Mitchel et. al., Inorg. Chem., 40: 2751 (2001); Sano, Nature (London), 414: 506 (2001); Shvartsburg, Phys. Chem. 103: 5275 (1999); and Li et al., Chem. Phys. Lett. 335: 524 (2001).

In general, fullerenes are hydrophobic and sparingly soluble in many solvents. See, e.g., Braun et al., Fullerenes, Nanotubes and Carbon Nanostructures, 15; 311-314 (2007). However, a variety of procedures for functionalizing fullerenes are known in the art, and some of the derivative fullerenes are water soluble. See, e.g., U.S. Pat. No. 5,648,243 to Chiang; U.S. Patent Application Publication Nos. 2008/0004345 and 2004/0044062; Jensen et al., Bioorganic & Medicinal Chemistry, 4:767-79 (1996); Da Ros et al., Croatica Chemica Acta CCACAA 74:743-55 (2001); Wilson, “Perspectives in Fullerene Nanotechnology,” Osawa, ed., (Kluwer Academic Publishers, Dorcrecht, Netherlands, 2000); Syrensky, et al., Kopf Carrier #63, (David Kopf Instruments Tujunga, California, September 2006); Y. L. Lai and L. Y. Chiang, J. Autonomic Pharmacol., 17:229 (1997); Schinazi et al., Proc. Electrochem. Soc., 97:10, (1997); Lai et al., World J. Surg., 24:450 (2000); Jin et al., J. Neuroscience Res., 62:600 (2000); Huang et al., Free Radical Biol. Med., 30:643 (2001); Chi et al., “Perspectives of Fullerene Nanotechnology,” pp 165-183, E. Osawa ed., (Kluwer Academic Publisher, Great Britain, 2002); Dugan et al., P.N.A.S. 94:9434-39 (1997); Dugan et al., Parkinsonism & Related Disorders 7:243-46 (2001); Quick et al., Neurobiol of Aging (electronic publication 2006); Kato et al., Chem & Biodiv., 2:1232-1241 (2005); Georgakilas et al, Proc. Nat. Acad. Sci. 99; 5075-5080 (2002).

Incorporation of fullerenes into lipid vesicles has also been studied (see, e.g., Bensasson et al., Journal of Physical Chemistry, 98:3492-3500 (1994); Hirsch et al., Angewandte Chemie International Edition 39:1845-1848 (1999); U.S. Pat. No. 7,070,810; Felder, et al., Helv. Chim. Acta, 85: 288-319 (2002).

Fullerenes can also be modified at their surface to present specific biologically active groups, such as lectins or antibodies. See, e.g., U.S. Patent Application Publication No. 2005/0043787; U.S. Pat. No. 5,310,669. Certain chemically modified fullerenes are commercially available. See, e.g., BuckyUSA, Houston, Tex. and American Dye Source, Inc., Quebec, Canada.

Fullerenes and derivatives of fullerenes have been proposed as free radical scavengers. See, e.g., Haddon, J. Am. Chem. Soc. 112:3389 (1990); U.S. Pat. No. 5,648,243 to Chiang, U.S. Patent Application Publication No. 2003/0162837 by Dugan; U.S. Pat. No. 7,163,956 to Wilson; Kepley, J. Immunol. 179:665 (2007).

### SUMMARY

Disclosed herein are methods of treating pruritus comprising administering a therapeutically effective amount of fullerenes to a subject in need thereof.

Pruritus can be caused and/or aggravated by dry skin, allergic reactions, allergies, insect bites and stings, insect allergies, tick bites, flea bite, worm allergies (e.g., threadworm, etc.), irritating chemicals, parasites (e.g., pinworms, scabies, lice, etc.), pregnancy, rashes, reactions to medicines, rash, itchy rash, skin inflammation, blisters, hives, eczema, contact dermatitis, poison oak, poison ivy, shingles, fungal and/or bacterial infection, lichen simplex, pityriasis, rosea, lichen sclerosis et atrophicus, nodular prurigo, vulval itch, chicken pox, measles, itch, anal itch, genital itch, reaction to medication, food allergy, jaundice, leukemia, polycythemia, kidney disease, hypothyroidism, hyperthyroidism, senile pruritus, fibers (e.g., fiber glass), psoriasis, eczema (atopic dermatitis), dermatitis (e.g., dermatitis herpetiformis, solar dermatitis, etc.), sun burn, jaundice, liver diseases, uremia, polycythemia vera, lymphoma, Hodgkin's lymphoma, leukemia, jock itch, feminine itch, and/or psychogenic itch.

### BRIEF DESCRIPTION OF THE DRAWINGS

FIG. 1 illustrates non-limiting examples of synthetically modified fullerenes with any combination of hydrophilic, lipophilic, or amphiphilic moieties.

FIG. 2 illustrates non-limiting examples of synthetically modified fullerenes with any combination of hydrophilic, lipophilic, or amphiphilic chemical moieties.

FIG. 3 illustrates additional non-limiting examples of synthetically modified fullerenes with any combination of hydrophilic, lipophilic, or amphiphilic chemical moieties.

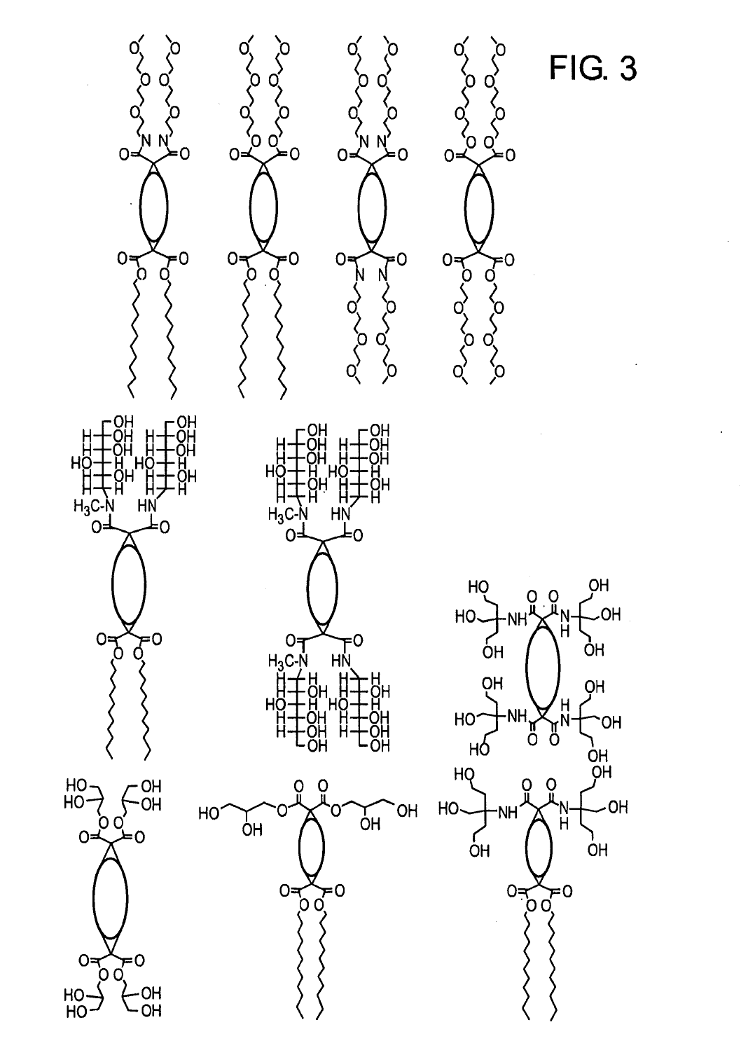

FIG. 4 illustrates additional non-limiting examples of synthetically modified fullerenes with any combination of hydrophilic, lipophilic, or amphiphilic chemical moieties.

FIG. 5 illustrates itch response in mice when fullerenes were injected prior to substance P.

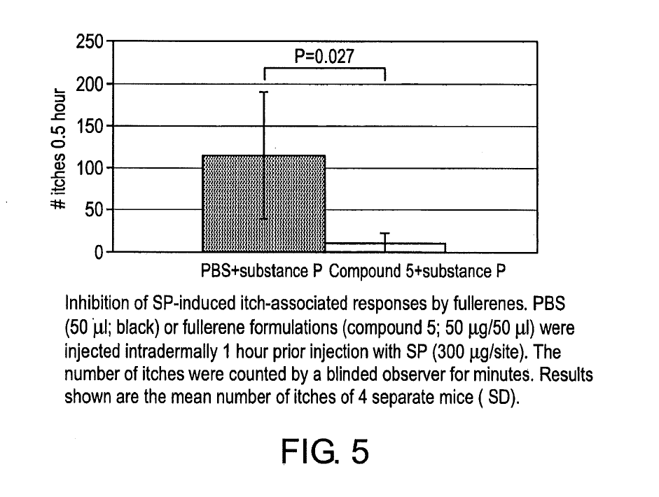

FIG. 6 illustrates non-limiting examples of water soluble and water insoluble fullerenes.

### DETAILED DESCRIPTION

In accordance with this detailed description, the following abbreviations and definitions apply. It must be noted that as used herein, the singular forms “a”, “an”, and “the” include plural referents unless the context clearly dictates otherwise. Thus, for example, reference to “compounds” includes a plurality of such compounds and reference to “the dosage” includes reference to one or more dosages and equivalents thereof known to those skilled in the art, and so forth.

The publications discussed in this disclosure are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed.

Unless otherwise stated, the following terms used in the specification and claims have the meanings given below:

“Pruritus” and “itch” as used herein are interchangeable and refer to an unpleasant cutaneous sensation which provokes the desire to scratch. Pruritus can occur in various levels of severity-mild pruritus, acute pruritus, and chronic pruritus, etc. Mild and acute pruritus, like pain, can serve a protective function, but chronic pruritus can have a significant negative impact on the quality of life of a subject. Pruritus may be widespread or localized on a subject's body.

Pruritus, especially acute and chronic pruritus, can negatively affect patients profoundly. Several studies have shown that people with chronic pruritus often have difficulty sleeping, problems concentrating, lowered sex drive, and suffer from depression. In addition to discomfort associated with pruritus, one of the major concerns related to chronic pruritus is the effect of constant scratching on the skin. Scratching an itchy area leads to increased inflammation, which causes further itching and scratching. This cycle of itching and scratching is sometimes referred to as the itch-scratch cycle. In addition, trauma to healthy-appearing skin from scratching can lead to the development of new lesions.

Typical symptoms of irritation include itching, stinging, burning, tingling, tightness, erythema (redness), or edema (swelling). The irritation response may be due to the direct effect on the skin of certain topical product chemicals or to a response by the immune system directed toward the chemicals alone or in combination with skin components (e.g., allergic dermatitis).

The sensation of itch is one of the most common skin problems experienced by humans and animals. Itch can be defined as a sensation which provokes the desire to scratch the site from which the sensation originates. All skin contains sensory nerves which transmit itch in response to chemical irritation, environmental exposure or disease processes. Although the precise population of itch producing nerves have not been identified, the thinnest, unmyelinated nerve population, termed type C nociceptive neurons are thought to be the most important in producing the sensation. Itch: Mechanisms and Management of Pruritus. Jeffrey D. Bernhard. McGraw-Hill, Inc. (San Francisco, 1994), pp. 1-22. The itch-producing nerves of the skin can be considered to be a “final common pathway” for the many irritating conditions which are ultimately sensed as itch including chemical exposure, environmental exposure (such as that which produces dry, itchy skin) and disease processes such as atopic dermatitis. Many chemical substances are able to produce itch when topically applied to the skin. No matter what the ultimate cause of itch, the sensation experienced is the same and provokes the desire to scratch.

Itch is a well known sensory state associated with the desire to scratch. As with pain, itch can be produced by a variety of chemical, mechanical, thermal or electrical stimuli. In addition to the difference in the sensory quality of itch and pain, they also differ in that (1) itch, unlike pain, can only be evoked from the superficial layers of skin, mucosa, and conjunctiva, and (2) itch and pain usually do not occur simultaneously from the same skin region; in fact, mildly painful stimuli, such as scratching, are effective in eliminating itch. In addition, the application of histamine to skin produces itch but not pain. Itch and pain are further dissociated pharmacologically: itch appears to be insensitive to opiate and non-steroidal anti-inflammatory drug (NSAID) treatment, both of which are effective in treating pain.

Although itch and pain are of a class in that both are modalities of nociception transmitted by small unmyelinated C fibers, evidence that itch is not just a variety of low-threshold pain is overwhelming. Itch leads to the reflex or urge to scratch; scratching leads to removal of the epidermis which eliminates itch but causes pain. Analgesics, particularly opioids, relieve pain but often cause itch. See, e.g., Bernhard J. D., Itching in the Nineties, J. Am. Acad. Derm., 24:309-310 (1991). There can be no doubt that itching is of eminent clinical importance; many systemic and skin diseases are accompanied by persistent or recurrent itch attacks. Current knowledge suggests that itch has several features in common with pain but exhibits intriguing differences as well. See, e.g., W. Magerl, IASP Newsletter, pp. 4-7 (September/October 1996).

Physiologically, there is evidence that substance P released from nociceptor terminals can cause the release of histamine from mast cells. Activation of mast cells, with release of the pruritogen histamine, occurs in immediate type hypersensitivity diseases, such as anaphylactic reactions and urticaria.

Without wishing to be bound by theory, it seems that pruritus occurs in two phases—(i) an itch identified with a local site, and (ii) a subsequent more diffused area of “itchy skin” that can be stimulated to intense pruritus by a light touch—for example, by fabric brushing against the skin. The dual-phase sensation appears to be similar to pain: (1) a sharp, relatively short-lived intense pain at the site of injury, and (2) a later, more generalized area of pain that can be intensified by pressure against the skin.

Further, without wishing to be bound by theory, pruritus seems to be associated with the spinal cord circuits involving so-called “A” and “C” fibers in the skin. The A and C fibers can give rise to free nerve endings in skin that respond to mechanical stimulus such as pressure, heat, cold, and certain chemicals. The responses seem to be associated with pain. Those fibers that respond to histamine (a potent itch-producing chemical) may contribute to the sensation of itching. Other itch-producing chemicals include opiates, papain, and members of the chemical group called kinins.

The apparent similarities between pruritus and pain have led a number of investigators to study specific “pruritus nerve fibers” in the skin that may parallel specific pain pathways. However, neither A nor C fibers seem to be the “itch fibers”. Without wishing to be bound by theory, scratching seems to send signals to the spinal cord “control box,” overloading the circuits thereby inhibiting the response of spinal cord nerve cells to signals from A and C fibers.

Without wishing to be bound by theory, when stimulated by a pruritogen, a subset of specialized C-fibers, originating superficially in the skin, conveys impulses to the dorsal horn of the spinal cord and then via the spinothalamic tract to the thalamus, and on to the somatosensory cortex. These C-fibers are anatomically identical to those associated with the mediation of pain but functionally distinct. In fact, the fibers are typically considered to be pain fibers, and itch has been categorized as a form of pain.

The most common type of C-fiber is the mechanical and heat nociceptor (polymodal nociceptor or CMH unit). These are either insensitive to histamine (see below) or only weakly activated by it. C-fibers which mediate itch comprise about 5% of the afferent C-fibers in human skin nerves; they respond to histamine and other pruritogens but are insensitive to mechanical stimuli. Thus, it is theorized that the sensation of pruritus is transmitted through the C-fibers in the skin to the dorsal horn of the spinal cord, and then, via the spinothalamic tract to the cerebral cortex of the brain for processing.

There are six general categories of pruritus: dermatologic, systemic, neurogenic, psychogenic, mixed, and other. Probably the most widely known dermatological mechanism of itch is through the release of histamine from mast cells and basophils. This can occur immunologically when allergens bind to immunoglobin E found on the surface of mast cells and basophils which leads to the release of histamine prestored in granules. Histamine can also be released from mast cells and basophils through non-immunologically mediated mechanisms such as cold, stress, and certain chemicals described below. Histamine also increases surface wound blood flow, which would explain the raised red surface usually present on the chronically itching wound. Histamine directly stimulates histamine type 1 (H1)-receptors on itch-specific neurons and can induce the classic wheal-and-flare response. While the wheal is a response to H1-receptor stimulation, the flare is the result of the secondary release of vasoactive substances from collateral axons. The wheal-and-flare response is specific for histamine-mediated itch. Histamine is the mediator for itch in several conditions, including: (i) most forms of urticaria; (ii) insect bite reactions; (iii) cutaneous mastocytosis; and (iv) drug rashes, e.g., antibiotics. The involvement of histamine is confirmed by the antipruritic effect of low-sedative H1-antihistamines in these conditions. The main source of histamine in the skin is the dermal mast cell from which it is released by mast cell degranulation. See, e.g., Paus et al., “Frontiers in pruritus research: scratching the brain for more effective itch therapy,” J. Clin. Invest., 116(4):1174-1185 (May 2006).

Several other compounds can induce the itch response. Acetylcholine stimulates histamine-sensitive and histamine-insensitive neurons. Serotonin (5-hydroxytryptamine, 5HT) can cause itch by both peripheral and central mechanisms. Peripherally, it acts indirectly through the release of histamine from dermal mast cells. Prostaglandins are not themselves pruritogenic, but potentiate itching caused by histamine and probably other mediators.

Cytokines are also major itch factors. For example several hours after interleukin-2 (IL2) is injected intradermally in both atopic and non-atopic subjects, itch and erythema occur and last 2-3 days. When given intravenously with cytotoxic drugs in the treatment of malignant melanoma, IL-2 causes intense itch.

Neuropeptides such as Substance P potentiate itch. Substance P is a short-chain polypeptide that functions as a neurotransmitter and as a neuromodulator. It belongs to the tachykinin neuropeptide family. It is unclear if substance P induces histamine release from mast cells to induce itch or if it induces itch by itself. For example substance P can directly induce human skin mast cells to release histamine (see, e.g., Oskeritzian et al., “Surface CD88 functionally distinguishes the MC from the MC type of human lung mast cell,” J. Allergy and Clin. Immunol., 115(6):1162-1168 (2005)) and elicits an itch response in both humans and mice (see, e.g., Hagermark et al., “Flare and Itch Induced by Substance P in Human Skin,” J. of Investigative Dermatology, 71:233-235 (1978); Barnes et al., “Plasma histamine levels following atracurium,” Anaesthesia, 41(8):821-824 (1986); Kuraishi et al., “Scratching behavior induced by pruritogenic but not algesiogenic agents in mice,” European Journal of Pharmacology, 275(3):229-233 (1995); Andoh et al., “Substance P Induction of Itch-Associated Response Mediated by Cutaneous NK1 Tachykinin Receptors in Mice,” J. of Pharmacology and Experimental Therapeutics, 286(3):1140-1145 (1998)). However, in mice intradermal injection of substance P itch-associated response is not inhibited by antihistamines. See, e.g., Andoh et al., “Involvement of Leukotriene B4 in Substance P-Induced Itch-Associated Response in Mice,” J. of Investigative Dermatology, 117:1621-1626 (2001)).

While it is unclear how substance P induces itch in mice and humans, substance P seems to have ubiquitous effects on many cell types and appears to influence a central point in the itch signaling cascade (C-fibers) leading from initial epidermal stimulus to the recognition of that stimulus in the brain. Without wishing to be bound by theory, it is believed that fullerenes may act at this central control point which can influence the many stimuli described above that lead to itch.

Urticarial eruptions are distinctly pruritic and can involve any portion of the body, and have a variety of causes beyond hypersensitivity, including physical stimuli such as cold, solar radiation, exercise and mechanical irritation. Other causes of pruritus include: chiggers, the larval form of which secretes substance that creates a red papule that itches intensely; secondary hyperparathyroidism associated with chronic renal failure; cutaneous larva migrans, caused by burrowing larvae of animal hookworms; dermal myiasis, caused by maggots of the horse botfly, which can afflict horseback riders; onchocerciasis (“river blindness”) caused by filarial nematodes; pediculosis, caused by lice infestations; enterobiasis (pinworm) infestations, which afflict millions of Americans, particularly school children; schistosome dermatitis (swimmer's itch); psoriasis; poison ivy; and asteatotic eczema (“winter itch”). The role of histamine or other endogenous pruritogens in mediating itch associated with these and other pruritic conditions, such as atopic dermatitis, its not yet well established. For atopic dermatitis, in particular, it appears that itch is not inhibited by antihistamines, but by cyclosporin A, a drug which inhibits the production of cytokines which have been proposed as potential pruritogens.

Pruritus is generally a common symptom of skin diseases. Systemic diseases and other conditions may also cause pruritus. For example, acute or chronic pruritus can be a common and disabling problem in many burn victims with healed burn wounds. In addition pruritus can be caused and/or aggravated by, but not limited to, the following: dry skin, allergic reactions, allergies, insect bites and stings, insect allergies, tick bites, flea bite, worm allergies (e.g., threadworm, etc.), irritating chemicals, parasites (e.g., pinworms, scabies, lice, etc.), pregnancy, rashes, reactions to medicines, rash, itchy rash, skin inflammation, blisters, hives, eczema, contact dermatitis, poison oak, poison ivy, shingles, fungal and/or bacterial infection, lichen simplex, pityriasis, rosea, lichen sclerosis et atrophicus, nodular prurigo, vulval itch, chicken pox, measles, itch, anal itch, genital itch, reaction to medication, food allergy, jaundice, leukemia, polycythemia, kidney disease, hypothyroidism, hyperthyroidism, senile pruritus, fibers (e.g., fiber glass), psoriasis, eczema (atopic dermatitis), dermatitis (e.g., dermatitis herpetiformis, solar dermatitis, etc.), sun burn, jaundice, liver diseases, uremia, polycythemia vera, lymphoma, Hodgkin's lymphoma, leukemia, jock itch, feminine itch, and psychogenic itch.

The following list of exemplary conditions can cause symptoms of pruritus: acute kidney failure, allergies, anaphylaxis, arthritis, athlete's foot, autoimmune hepatitis, blepharitis, candidiasis, cercarial dermatitis, chickenpox, chilblain, cholangitis, cholecystitis, chronic kidney failure, ciguatera poisoning, cirrhosis of the liver, cutaneous mastocytosis, decompression sickness, dermatitis, dermatitis herpetiformis, diabetes, drug allergies, dry skin, eczema, food allergies, heat rash, Hodgkin's disease, hyper-IgE syndrome, hyperthyroidism, hypothyroidism, jaundice, lichen sclerosis, lymphoma, mastocytosis, molluscum contagiosum, mycosis fungoides, non-Hodgkin's lymphoma, osteoarthritis, pancreatic cancer, pediculosis, pityriasis rosea, polycythemia, porphyria, primary biliary cirrhosis, primary sclerosing cholangitis, psoriasis, rabies, scabies, schistosomiasis, Sjogren's syndrome, slap-cheek syndrome, type 2 diabetes, Wiskott-Aldrich Syndrome, and yaws.

Exemplary causes of localized pruritus include eczema, contact dermatitis, poison oak, poison ivy, insect bite, insect sting, parasites, scabies, lice, tick bite, shingles. Examples of diseases that cause an itchy rash include: hives, blisters, eczema, lice, scabies, insect bite, insect sting, fungal infection, lichen simplex, pityriasis, rosea, lichen sclerosis et atrophicus, nodular prurigo, vulval itch, chicken pox, measles. Adverse reactions to medications include certain medications, see also causes of itchy rash, itch, anal itch, vulval itch, genital itch. Some possible causes of itching all over include allergic reaction (type of an adverse reaction), reaction to medication, food allergy, insect allergy, jaundice, leukemia, polycythemia, kidney disease, dry skin, hypothyroidism. Some diseases that cause itch without a rash include lice, scabies, insect bite, threadworm, flea bite, senile pruritus, fiber glass fibers, pregnancy, dermatitis herpetiformis, jaundice, liver diseases, uremia, polycythemia vera, lymphoma, Hodgkin's lymphoma, leukemia, psychogenic itch, and certain medications. The methods of treating pruritus in a subject in need thereof are disclosed herein. The methods described herein disclose treating pruritus that is (i) immunologically-mediated, (ii) histamine-induced, (iii) non immunologically-mediated, (iv) parasite-induced, (v) fungus and bacteria induced, (vi) induced by insect bites, (vii) induced by plant derived pruritogens, and/or (viii) induced by poison ivy.

Allergic diseases such as atopic dermatitis have caused severe problems in recent years. Atopic dermatitis occurs as dry lichen like eczema, and is an intractable disease accompanying severe itch. While little is known about the mechanism of the onset of eczema of the atopic dermatitis, it has been made clear that the disease is related to allergic reactions type I and type IV. A type I allergy is an immediate allergy mainly related to IgE antibodies, wherein chemical transmitters such as histamine and leukotriene, and enzymes are released upon reaction of allergenic substances (allergens) with IgE on mast cells and basophils to cause inflammatory reactions on the skin. However, type I allergies are considered not to be directly related to lesions of eczema caused by the atopic dermatitis. A type IV allergy is called as a delayed allergy or cell mediated allergy that causes an inflammatory reaction related to lymphocytes, and the onset of the disease takes 24 to 72 hours after sensitization.

Lice carry a wide variety of bacteria on their exterior surfaces, and their fecal matter transmits disease when it enters the puncture wounds inflicted during feeding. As the lice feed on human skin, they inject their digestive juices into the skin. These materials, as well as the puncture wound itself, cause pruritus, which is painful to the animal host. Therapy that simply kills the louse leaves subcutaneous or intradermal residues that continue to itch for a significant time after the infestation is extinguished. Furthermore, scratching during and after the episode frequently leads to painful excoriation.

Plant irritants are exemplary of the irritants which come into contact with the skin and lead to itching. For example, itching is commonly associated with contact dermatitis induced by irritants present in plants of the Anacardiaceae and Ginkgoaceae families. Included among these plants are the Toxicodendrons, e.g., poison ivy (T. radicans), Eastern poison oak, (T. quercifolium), poison sumac (T. vernix), and Western poison oak (T. diversilobum). Also included are manzanillo (Rhus striata), Japanese lac (R. verniciflua), Mango tree (Mangifera indica), India ink tree, (Semicarpus anacardum), ginko tree (Ginko biloba L.), and the cashew tree (Anacardium occidentale).

One such group of plant-borne irritants is a mixture of alkyl-catechols known as the urushiols. Urushiols are present in, e.g., poison ivy and Japanese lac, and are known to induce the contact dermatitis commonly known as “poison ivy” rash. One to two million people are said to be afflicted with varying degrees of poison ivy rash each year. It has been estimated that as many as 150,000 working days are lost annually to urushiol induced contact dermatitis. Vietmeyer, N., Smithsonian, 16(5):89-95 (August 1985).

The process leading to urushiol-induced contact dermatitis, or poison ivy rash, is reported to be as follows. The mechanism of action of urushiols in inducing contact dermatitis has been described in the literature. See, e.g., Lepoittevin, J-P. and Benezra, C., “Allergic contact dermatitis caused by naturally occurring quinones,” Pharm. Weekbl [Sci] (Netherlands), 13(3):119-122 (1991). It is believed that the mechanism of action of the urushiols may be general to many irritants that lead to itching. Urushiols are transmitted from the plant to the skin and are oxidized in vivo to ortho-quinones. The ortho-quinones are thought to interact with proteins and form quinone-protein conjugates which act as the actual antigens. Next, the conjugates are reported to be presented to inexperienced T-lymphocytes by epidermal macrophages. The exposed lymphocytes can develop into immunologically competent lymphocytes which are capable of reacting to subsequent exposure to the urushiols. At this point a mammal is said to be sensitized to the urushiols. The subsequent exposure of these competent lymphocytes to urushiol (ortho-quinone)-protein conjugates is thought to lead to the release of lymphokines and the manifestation of urushiol induced contact dermatitis—the poison ivy rash.

Poison ivy rash starts with a reddening of the skin and itching. The itching intensifies with time and watery blisters appear. The itching typically causes afflicted mammals to scratch the affected area. This can delay healing and can spread urushiols to a wider area of the skin or other parts of the body.

The widespread occurrence of poison ivy induced dermatitis has led to the proposal of many treatments to prevent or alleviate the symptoms of the poison ivy rash. Methods reported to prevent the rash include the use of desensitizing materials, urushiol absorbing materials, and forming protective layers on the skin to block irritants. Reportedly useful methods for alleviating symptoms include applying plant extracts, corticosteroids, and enzymes including catechol oxygenase or p-diphenol oxidase, washing irritants from the skin with polyglycol ethers, thymopentin therapy and the use of topical anesthetics. (Examples of reportedly useful methods of treatment to prevent or alleviate symptoms of contact dermatitis can be found in U.S. Pat. Nos. 5,086,075; 5,049,580; 5,036,050; 5,017,361; 5,011,689; 4,861,584; 4,738,956; 4,344,965; 4,428,965; 4,259,318; 4,199,575; 4,160,819; 4,144,319; 4,141,966; 4,137,301; 4,112,067; 4,002,737 and 3,974,281; Canadian Patent No. 1032473; and European Patent No. 0 311 963 A1, all of which are hereby incorporated by reference.)

Many ingredients used in topical products are known irritants or are potentially irritating, especially to people with sensitive skin. These irritating ingredients include fragrances, preservatives, solvents, propellants and many other ingredients that might otherwise be considered inert components of the products. Additionally, many topical product active ingredients, including chemicals that may also be classified as drugs, produce irritation when applied to the skin. These include, but are not limited to, such ingredients as exfoliants and skin cell renewal agents, anti-acne drugs, antiperspirant compounds, antihistamines, anti-inflammatory agents, skin protective agents, insect repellent chemicals, sunscreens and many others. Where more than one chemical irritant is present, their irritating effects may be additive. Furthermore, chemical ingredients may react with one another, or in the environment of the skin, to form new chemicals which are irritating. The vehicles in which the active drug ingredients are formulated may also produce irritation in some subjects, especially in drugs such as topical corticosteroids.

In addition to chemicals which directly trigger skin irritation, some chemicals indirectly cause the skin to become more sensitive to other chemicals or environmental conditions which would not normally cause irritation. Many chemicals which act as skin exfoliants such as retinoids (e.g., tretinoin, retinol and retinal), carboxylic acids including α-hydroxy acids (e.g., lactic acid, glycolic acid), β-hydroxy acids (e.g., salicylic acid), α-keto acids, acetic acid and trichloroacetic acid, 1-pyrrolidone-5-carboxylic acid, capryloyl salicylic acid, α-hydroxy decanoic acid, α-hydroxy octanoic acid, gluconolactone, methoxypropyl gluconamide, oxalic acid, malic acid, tartaric acid, mandelic acid, benzylic acid, gluconic acid, benzoyl peroxide and phenol, among others, may cause the skin to become more sensitive to irritation triggered by other topically-applied chemicals such as moisturizers, sunscreens, fragrances, preservatives, surfactants (e.g., soaps, shaving cream) and other topical products. Exfoliants and other ingredients may also increase the skin's sensitivity to environmental conditions such as sunlight, wind, cold temperature and dry air, or may exacerbate the irritation attributable to a pre-existing skin disease.

Conversely, environmental influences may themselves increase the skin's sensitivity to chemicals in topical products by reducing the skin's barrier function. The barrier function acts to minimize absorption or passage of potentially irritating chemicals through the outer dead cell layer into the living skin tissue. Extremes of humidity, for example, can greatly increase irritation from topically-applied products. A very common condition due to low humidity is termed “winter itch” in which the very low humidity characteristics of many cold climates (particularly when accompanied by indoor heating) or long exposure to refrigerated air from air conditioners in the summer produces itchy skin—especially in older people—which can exacerbate the irritating effects of topical products. Additionally, soaps, detergents, cleansing products, shaving creams, alcohol and other products which remove some of the skin's protective lipids and/or secretions may increase the skin's permeability and sensitivity to topically-applied chemicals which would otherwise not produce irritation. Normal processes such as sweating may also increase the ability of irritant materials, such as antiperspirants, deodorants or sunscreens, to penetrate the skin through pores or glands, thus exacerbating the potential for irritation. Exposure of the skin to high humidity environments or liquids may also increase the ability of potential irritants to penetrate the skin. Similarly, the skin may become sensitized or inflamed due to infection, shaving abrasion, repeated or excessive washing or bathing, sun exposure, or other mechanical abrasion or injury, resulting in sensory irritation responses upon subsequent application of underarm deodorants, after-shaves or other topical products.

In addition to chemical and environmental causes of skin irritation, many people have an inherent sensitivity or genetic predisposition to skin irritants. People with respiratory allergies, for example, tend to have excessively dry skin which facilitates increased absorption of potentially irritating chemicals. The excessively dry skin which accompanies atopic dermatitis, for example, predisposes patients with this condition to irritation from many topically-applied products. Other skin diseases and conditions such as allergic or non-allergic contact dermatitis, psoriasis, eczema, candida albicans, post-herpetic neuralgia, infectious diseases manifested by, for example, sore throat or skin lesions, insect bites and the like produce intrinsic irritation which may be exacerbated by application of topical products. Many other individuals exhibit sensitive skin as a condition that is not related to an identifiable skin disease.

“Pruritogen” refers to any substance that causes itch in an individual. Examples of pruritogens include urushiol. See above discussion for more information regarding the causes of pruritus.

“Fullerene” or “fullerene molecule” as used herein refers to any member of the fullerene family of carbon cage molecules. Fullerenes are generally carbon structures formed of five and six membered rings arranged so that the rings form a closed geodesic sphere or spheroid held together by a combination of single and double carbon:carbon covalent bonds. The fullerenes in this disclosure can be defined by the formula: C₂ₛ wherein s is greater than or equal to 30, such as from about 30 to about 200 or from about 30 to about 100. For example, the fullerenes include C₆₀, C₇₀, and similar molecules that range in molecular weight from C₆₀ up to C₈₄, C₉₀, and larger such molecules, with shapes ranging from spheroidal to ellipsoidal, elongated and other shapes, and including not only single-walled but also multi-walled cages consisting of stacked or parallel layers. The fullerenes may be unmodified or underivatized. Alternatively, the fullerenes may enclose one or more atoms such as metal atoms, or other small chemical groups, inside the carbon cage; such fullerenes are sometimes called endohedral fullerenes. Fullerenes, as used herein, also includes structures with chemical functional groups attached to the surface of the carbon cage. The functional groups can be covalently bound to the carbon cage via opening carbon:carbon double bonds. Fullerenes also include other structural variants, derivatives, and/or modified or functionalized fullerenes as described herein and/or as known in the art. The fullerenes can be synthetic or naturally-occurring. Synthetic fullerene molecules can be prepared in a laboratory by known methods (see, e.g., U.S. Pat. No. 5,177,248 and Krätschmer et al., Chem. Phys. Lett., 170, 167-170 (1990)) or can be purchased commercially.

In one embodiment, the fullerenes are water soluble, meaning the fullerenes distribute more or less uniformly in an aqueous solution and do not significantly precipitate. Water soluble fullerenes are known in the art as described above, and can be synthesized for example by attaching one or more hydrophilic chemical groups to the surface of the carbon cage. Suitable hydrophilic chemical groups include hydroxyl or polyhydroxyl groups and N-ethylpolyamino groups. Non-limiting examples of water soluble fullerenes include C₆₀(OH)ₙ, C₆₀(NH—CH₂—CH₃)ₙ, and C₇₀-tetraglycolic acid (compound 7 or TGA). Many other examples of water-soluble fullerenes are known and can involve the addition of one or more charged groups such as phosphates, sulfates, ammonium, carboxylates, or other charged groups; or hydrophilic groups such as hydroxyl and polyhydroxyl groups; and carbohydrates, peptides, proteins, and DNA.

In another embodiment, chemical groups such as amphiphilic or lipophilic groups can be attached to the carbon cage instead of or in combination with hydrophilic chemical groups.

“Fullerene” or “fullerene molecule” as used herein refers to certain synthetically modified fullerene molecules as described herein, including amphiphilic or lipophilic synthetically modified fullerenes of the formula Zₘ—F—Yₙ; and hydrophilic or amphiphilic synthetically modified fullerenes of the formula Z′ₘ—F—Y′ₙ. The fullerenes comprise closed cages of 60 to 200 carbon atoms which may also include chemical moieties attached to the exterior and/or incorporated within the cage.

The amphiphilic or lipophilic synthetically modified fullerene molecules are described in copending U.S. patent application Ser. No. 2/073,230, U.S. Patent Application Publication No. 2008-0213324-A1 filed Mar. 3, 2008, entitled “AMPHIPHILIC OR LIPOPHILIC POLAR FUNCTIONALIZED FULLERENES AND THEIR USES,” the entire disclosure of which is incorporated by reference herein.

The amphiphilic or lipophilic and hydrophilic or amphiphilic synthetically modified fullerene molecules as described in the copending application include fullerenes that have an aspect ratio ≠1, with an equatorial band and two opposing poles, and comprise an adduct at one or both poles.

In one embodiment, the amphiphilic or lipophilic synthetically modified fullerene has the formula

Zₘ—F—Yₙ;

wherein F is a fullerene of formula Cₚ or X@Cₚ, the fullerene having two opposing poles and an equatorial region;

Cₚ represents a fullerene cage having p carbon atoms, and X@Cₚ represents such a fullerene cage having a chemical group X within the cage.

Z and Y are positioned near respective opposite poles of Cₚ;

m=1-5 and Z is a hydrophilic, lipophilic, or amphiphilic chemical moiety;

n=1-5 and Y is a lipophilic chemical moiety;

p=60-200 and p is an even number; and

X, if present, represents one or more metal atoms within the fullerene (F), optionally in the form of a trinitride of formula Gᵢ₌₁₋₃Hₖ₌₃₋ᵢN in which G and H are metal atoms.

In exemplary variations p is an even number between 60 and 120, with p=60-96 being more common and p=60 or p=70 being preferred. The synthetically modified fullerene can be arranged wherein each chemical moiety Z is composed of formula AᵣB in which A is a hydrophilic, lipophilic or amphiphilic chemical moiety, r=1-4, and B is a chemical linker connecting said A to the fullerene, and each chemical moiety Y is composed of formula DEᵥ in which E is a lipophilic chemical moiety, v=1-4, and D is a chemical linker connecting the lipophilic chemical moiety to the fullerene.

The amphiphilic or lipophilic synthetically modified fullerene can be a prolate ellipsoid shaped fullerene having a major axis such that said poles are located at opposing ends of the major axis of the prolate ellipsoid fullerene. Alternatively, the fullerene can be spheroid with opposing poles defined by an axis through opposing carbon rings. Z and Y can configured such that when the molecule is contacted with a lipid bilayer in an aqueous medium, the equatorial region of F is selectively located within or in close proximity to the phospholipid bilayer. The molecule can be configured so that in an extended configuration has an aspect ratio of about 2.1 to 15, and a diameter less than about 2 nm. Such configurations are preferred configurations for incorporation of the molecules into lipid bilayers.

In another embodiment, the amphiphilic or lipophilic synthetically modified fullerene molecule has the formula Z(Cₚ)Y wherein: p=60-200 carbons, preferably p=60 or 70; Y is a lipophilic moiety covalently connected to Cₚ, optionally through a linking group, at or near a pole thereof, and wherein Z is a lipophilic moiety, amphiphilic moiety, or a hydrophilic moiety covalently connected to Cₚ, optionally through a linking group, at or near a pole opposite to said Y; and, wherein said lipophilic moiety Y is capable of anchoring the synthetic fullerene molecule to a lipid membrane;

In another embodiment, the amphiphilic or lipophilic synthetically modified fullerene molecule has the formula Z(Cₚ)Y wherein: p=60-200 carbons, preferably p=60 or 70; Y is a lipophilic moiety covalently connected to Cₚ, optionally through a linking group, at or near a pole thereof, and wherein Z is a hydrophilic moiety covalently connected to Cₚ, optionally through a linking group, at or near a pole opposite to said Y; and, wherein said lipophilic moiety Y is capable of anchoring the synthetic fullerene molecule to a lipid membrane.

In another embodiment, the amphiphilic or lipophilic synthetically modified fullerene molecule has the formula Z(C₇₀)Y; wherein Y is a lipophilic moiety covalently connected to C₇₀, optionally through a linking group, at or near a pole thereof, and wherein Z is a lipophilic moiety, amphiphilic moiety, or a hydrophilic moiety covalently connected to C₇₀, optionally through a linking group, at or near a pole opposite to said Y; and, wherein said lipophilic moiety Y is capable of anchoring the synthetic fullerene molecule to a lipid membrane.

In another embodiment, the amphiphilic or lipophilic synthetically modified fullerene molecule has the formula Z(C₇₀)Y wherein: Y is a lipophilic moiety covalently connected to Cₚ, optionally through a linking group, at or near a pole thereof, and wherein Z is a hydrophilic moiety covalently connected to Cₚ, optionally through a linking group, at or near a pole opposite to said Y; and, wherein said lipophilic moiety Y is capable of anchoring the synthetic fullerene molecule to a lipid membrane.

In another embodiment the amphiphilic or lipophilic synthetically modified fullerene molecule can have the formula Zₘ—F—Yₙ wherein:

F is a fullerene of formula Cₚ having p=60-200 carbons, preferably p=60 or 70;

m=1-5 such that each Z is a group AᵣBₛ in which r=1-4, s=1-4, and A is one or more hydrophilic or charged group bonded to the fullerene through one or more linker B;

n=1-5 and each Y is a group DₜEᵥ in which t=1-4, v=1-4 and E is one or more lipophilic group bonded to the fullerene through one or more linker D; and,

X and Y are positioned at or near opposite poles of F.

In certain embodiments the amphiphilic or lipophilic synthetically modified fullerene has a geometrical configuration capable of causing the fullerene molecule to locate within phospholipid bilayers of a cell such that a radical scavenging zone near the equatorial band of the fullerene is situated within or in close proximity to the phospholipid bilayer.

A plurality of such synthetically modified fullerene molecules can be uniformly dispersed in phospholipids, such as in liposomes. The amphipathic fullerene molecules described herein do not generally form vesicles by themselves, but require membrane-forming phospholipids in mole ratios greater than 1:1 (lipid:fullerene adduct) to form vesicles.

The methods described herein also encompass hydrophilic or amphiphilic synthetically modified fullerenes of the formula

Z′ₘ—F—Y′ₙ;

wherein F is a fullerene of formula Cₚ or X@Cₚ, the fullerene having two opposing poles and an equatorial region;

Cₚ represents a fullerene cage having p carbon atoms, and X@Cₚ represents such a fullerene cage having a chemical group X within the cage;

Z′ and Y′ are positioned near respective opposite poles of Cₚ;

m=1-5 and Z′ is a hydrophilic, lipophilic, or amphiphilic chemical moiety;

n=1-5 and Y′ is a hydrophilic or amphiphilic chemical moiety;

p=60-200 and p is an even number; and

X, if present, represents one or more metal atoms within the fullerene (F), optionally in the form of a trinitride of formula Gᵢ₌₁₋₃Hₖ₌₃₋ᵢN in which G and H are metal atoms.

In exemplary variations p is an even number between 60 and 120, with p=60-96 being more common and p=60 or p=70 being preferred. The fullerene can be arranged wherein each chemical moiety Z′ is composed of formula A′ᵣB in which A′ is a hydrophilic, lipophilic or amphiphilic chemical moiety, r=1-4, and B is a chemical linker connecting said A′ to the fullerene, and each chemical moiety Y′ is composed of formula DE′ᵥ in which E′ is a hydrophilic or amphiphilic chemical moiety and, v=1-4, and D is a chemical linker connecting the chemical moiety Y′ to the fullerene.

In another embodiment, the hydrophilic or amphiphilic synthetically modified fullerene molecule has the formula Z′(Cₚ)Y′ wherein: p=60-200 carbons, preferably p=60 or 70; Y′ is a hydrophilic or amphiphilic moiety covalently connected to Cₚ, optionally through a linking group, at or near a pole thereof, and wherein Z′ is a hydrophilic or amphiphilic moiety covalently connected to Cₚ, optionally through a linking group, at or near a pole opposite to said Y′.

In exemplary embodiments, Z′ and Y′ are both amphiphilic; Z′ and Y′ are both hydrophilic; or one of Z′ and Y′ is amphiphilic while the other is hydrophilic. In other embodiments, Z′ is lipophilic and Y′ is hydrophilic or amphiphilic.

In another embodiment, the hydrophilic or amphiphilic synthetically modified fullerene molecule has the formula Z′(C₇₀)Y; wherein Y′ is a hydrophilic or amphiphilic moiety covalently connected to C₇₀, optionally through a linking group, at or near a pole thereof, and wherein Z′ is a hydrophilic or amphiphilic moiety covalently connected to C₇₀, optionally through a linking group, at or near a pole opposite to said Y′.

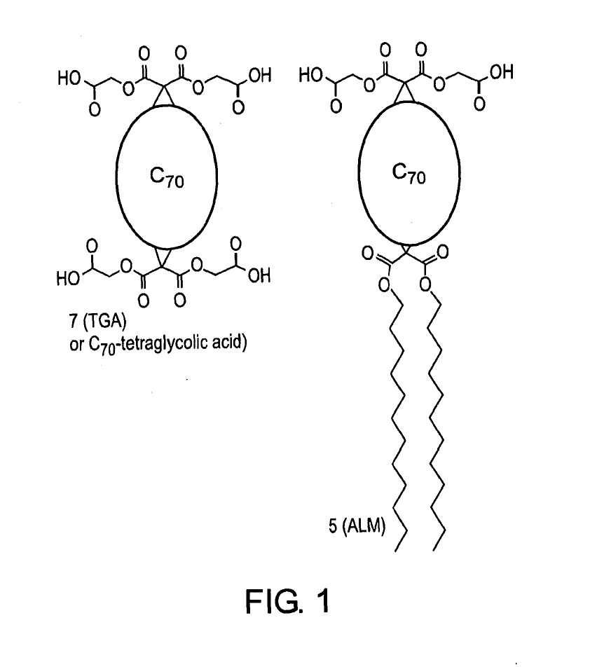

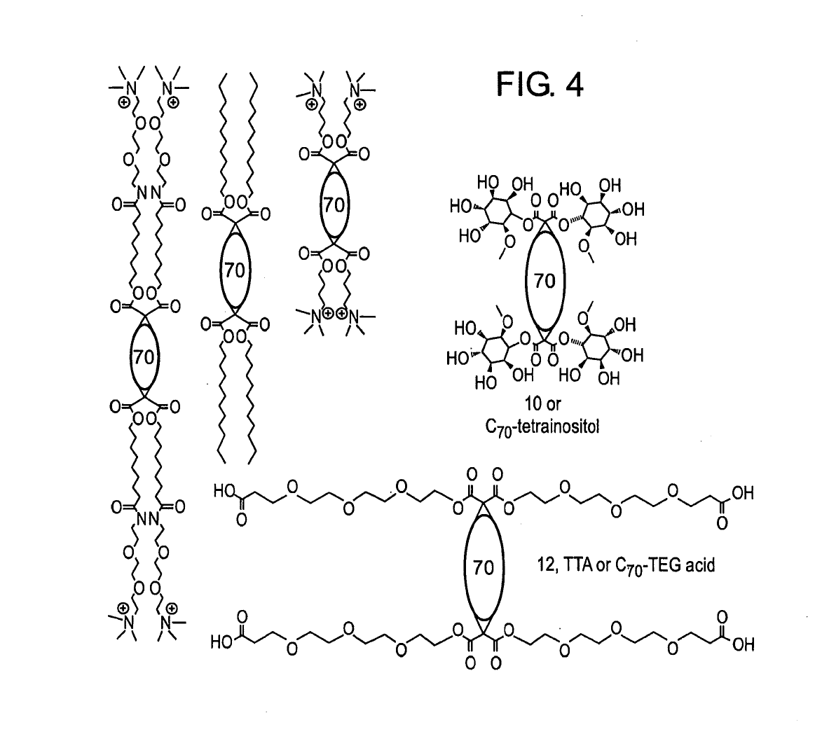

In certain embodiments, the fullerene comprises any one or more of the fullerenes set forth in the present figures. In an exemplary embodiment, the fullerene is one or more of compounds 5 (illustrated in FIG. 1), 7 (illustrated in FIG. 1), 10 (illustrated in FIG. 4), and 12 (illustrated in FIG. 4). In the present examples, compound 5 comprises C₇₀.

Suitable fullerenes are also described in the following co-pending PCT applications filed concurrently herewith: Attorney Docket No. 1034136-000062, entitled “USING FULLERENES TO ENHANCE AND STIMULATE HAIR GROWTH;” Attorney Docket No. 1034136-000064, entitled “FULLERENE THERAPIES FOR INFLAMMATION;” Attorney Docket No. 1034136-000065, entitled “METHOD FOR INHIBITING THE BUILD-UP OF ARTERIAL PLAQUE;” and Attorney Docket No. 1034136-000066, entitled “METHOD FOR TREATING WOUNDS BY ADMINISTERING FULLERENES;” the entire disclosures of which are incorporated by reference herein, and in U.S. Patent Application Nos. 61/071,756, filed May 15, 2008, entitled “NEW REACTIONS OF FULLERENES” and Ser. No. 12/073,231, filed Mar. 3, 2008, entitled “STEROID DERIVATIVES OF FULLERENES,” the entire disclosures of which are incorporated by reference herein.

The terms “treating,” “treatment,” and the like are used herein to generally mean obtaining a desired pharmacological and physiological effect, and refer to complete elimination as well as to any clinically or quantitatively measurable reduction in the condition for which the subject is being treated. “Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. More specifically, the fullerenes described herein which are used to treat a subject with pruritus are provided in a therapeutically effective amount to prevent the disorder (i.e., inhibit the onset or occurrence of the disorder and/or cause the clinical symptoms of the disorder not to develop in a mammal that may be exposed to or predisposed to the disorder but does not yet experience or display symptoms of the disorder); inhibit the disorder (i.e., arrest or reduce the development of the disorder or its clinical symptoms); or relieve the disorder (i.e., cause regression of the disorder or its clinical symptoms). Subjects in need of treatment include those already with pruritus as well as those in which pruritus is to be prevented.

A “subject in need thereof” refers to any subject or individual who could benefit from the method of treatment described herein. In certain embodiments, a subject in need thereof is a subject having pruritus (i.e., experiencing itching on a localized or widespread portion of the subject's body). The “subject in need thereof” refers to a vertebrate, preferably a mammal. Mammals include, but are not limited to, humans, other primates, rodents (i.e., mice, rats, and hamsters), farm animals, sport animals and pets. In one embodiment, the subject is a mammal such as a human. In certain embodiments, the methods find use in experimental animals, in veterinary application, and/or in the development of animal models for disease.

As used herein, the term “administering” or “introducing” fullerenes to a subject means providing the fullerenes to a subject. Methods of administering fullerenes to subjects are well known to those of ordinary skill in the art and include, but are not limited to, oral, intravenous, intramuscular, parenteral, or local administration. Modes of administration can also include delivery via a controlled release and/or controlled release drug delivery formulation and/or device.

“Sustained release” refers to release of a drug or an active metabolite thereof into the systemic circulation over a prolonged period of time relative to that achieved by oral administration of a conventional formulation of the drug.

“Controlled release” is a zero order release; that is, the drug releases over time irrespective of concentration. Single, multiple, continuous or intermittent administration can be effected.

“Orally delivered drugs” refer to drugs which are administered to an animal in an oral form, preferably, in a pharmaceutically acceptable diluent. Oral delivery includes ingestion of the drug as well as oral gavage of the drug.

“Therapeutic or prophylactic blood concentrations” refers to systemic exposure to a sufficient concentration of a drug or an active metabolite thereof over a sufficient period of time to effect disease therapy or to prevent the onset or reduce the severity of a disease in the treated animal.

“Optional” or “optionally” means that the subsequently described event or circumstance may, but need not, occur, and that the description includes instances where the event or circumstance occurs and instances in which it does not.

“Pharmaceutically acceptable salt” refers to pharmaceutically acceptable salts of fullerenes which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

As used herein, “pharmaceutically acceptable” means acceptable for use in the pharmaceutical and veterinary arts, compatible with other ingredients of the formulation, and not toxic or otherwise unacceptable commensurate with a reasonable benefit/risk ratio.

A “pharmaceutically acceptable carrier” or “diluent” includes any and all solvents, dispersion media, coatings, antibacterial and anti-fungal agents, isotonic and absorption delaying agents, and the like, compatible with pharmaceutical administration of a composition comprising ferritin-iron complexes. Examples of such carriers or diluents include, but are not limited to, water, saline, Ringer's solutions and dextrose solution. The volume of a pharmaceutical composition or formulation comprising fullerenes is based on the intended mode of administration and the safe volume for the individual patient, as determined by a medical professional.

The selection of carrier also depends on the intended mode of administration. Fullerenes of the present invention may be administered by any of a number of convenient means including, but not limited to systemic administration (e.g., intravenous injection, intraparenteral injection, inhalation, transdermal delivery, oral delivery, nasal delivery, rectal delivery, etc.) and/or local administration (e.g., direct injection into a target tissue, delivery into a tissue via cannula, delivery into a target tissue by implantation of a time-release material, or delivery through the skin via a topical composition such as a cream, lotion, or the like), delivery into a tissue by a pump, etc., orally, parenterally, intraosseously, in the cerebrospinal fluid, or the like. Further modes of administration include buccal, sublingual, vaginal, subcutaneous, intramuscular, or intradermal administration.

In some embodiments, a pharmaceutical composition or formulation comprising fullerenes is administered orally to a subject having pruritus. As used herein, “pharmaceutical composition” and “pharmaceutical formulation” are interchangeable. In another embodiment, a composition comprising fullerenes is injected directly into an affected area of the body of a subject having pruritus. In yet another embodiment, a composition comprising fullerenes is administered via a topical formulation applied to the skin proximal to an affected area of the body of a subject having pruritus.

A “therapeutically effective amount” or “pharmaceutically effective amount” means the amount of a fullerene that, when administered to a subject for treating pruritus. Thus a “therapeutically effective amount” is an amount indicated for treatment while not exceeding an amount which may cause significant adverse effects. The “therapeutically effective amount” will vary depending on the types of fullerenes to be administered, the severity of pruritus, and the age, weight, etc., of the subject to be treated. Methods for evaluating the effectiveness of therapeutic treatments are known to those of skill in the art.

Doses to be administered are variable according to the treatment period, frequency of administration, the host, and the nature and severity of the disorder. The dose can be determined by one of skill in the art without an undue amount of experimentation. The fullerenes are administered in dosage concentrations sufficient to ensure the release of a sufficient dosage unit into the patient to provide the desired treatment of pruritus. The actual dosage administered will be determined by physical and physiological factors such as age, body weight, severity of condition, and/or clinical history of the patient. The fullerenes may be administered to achieve in vivo plasma concentrations of the fullerenes of from 0.01 to 10,000 ng/cc. For example, the methods described in this disclosure may use compositions to provide from about 0.01 to about 100 mg/kg or from about 0.01 to about 10 mg/kg body weight/day of the fullerenes, such as about 30 mg/kg body weight/day of the fullerenes. It will be understood, however, that dosage levels that deviate from the ranges provided may also be suitable in the treatment of a given disorder. A practical dosage regimen is a schedule of drug administration that is practical for a patient to comply with. For human patients, a practical dosage regimen for an orally administered drug is likely to be an aggregate dose of less than 10 g/day.

The fullerenes may be in any form suitable for administration. Such administrable forms include tablets, buffered tablets, pills, capsules, enteric-coated capsules, dragees, cachets, powders, granules, aerosols, liposomes, suppositories, creams, lotions, ointments, skin patches, parenterals, lozenges, oral liquids such as suspensions, solutions and emulsions (oil-in-water or water-in-oil), ophthalmic liquids and injectable liquids, or sustained-release forms thereof. The desired dose may be provided in several increments at regular intervals throughout the day, by continuous infusion, or by sustained release formulations, or may be presented as a bolus, electuary or paste.

In various embodiments, a pharmaceutical composition or formulation comprising the fullerenes is prepared by admixture with one or more pharmaceutically acceptable carriers and/or excipients. Other additives and/or active ingredients may be added, if desired, to maximize the preservation of the fullerenes, to optimize a particular method of delivery, or to optimize treatment of pruritus in the subject in need thereof. In addition, according to other embodiments, the pharmaceutical composition or formulation comprising fullerenes may include other compositions comprising fullerenes as described herein in combination with other agents suitable for the treatment of pruritus.

In one embodiment, a kit for treating pruritus is disclosed. The kit comprises a pharmaceutical composition comprising fullerenes, wherein the pharmaceutical composition is administered in a therapeutically effective amount to a subject in need thereof.

The fullerenes may be formulated into a variety of compositions (i.e., formulations or preparations). These compositions may comprise any component that is suitable for the intended purpose, such as conventional physiologically acceptable delivery vehicles, diluents and excipients including isotonising agents, pH regulators, solvents, solubilizers, dyes, gelling agents and thickeners and buffers and combinations thereof. Pharmaceutical formulations suitable for use with the instant fullerenes can be found, for instance, in Remington's Pharmaceutical Sciences. Physiologically acceptable carriers are carriers that are nontoxic at the dosages and concentrations employed. Pharmaceutical formulations herein comprise pharmaceutical excipients or carriers capable of directing the fullerenes to the area where the subject in need thereof has pruritus. Suitable excipients for use with fullerenes include water, saline, dextrose, glycerol and the like.

In various embodiments, the fullerenes are administered to a subject in need thereof in the form of pharmaceutical compositions or formulations. These pharmaceutical compositions or formulations comprise fullerenes and can also include one or more pharmaceutically acceptable carriers or excipients. The excipient is typically one suitable for administration to human subjects or other mammals. In making the compositions of this disclosure, the active ingredient (i.e., fullerenes) is usually mixed with an excipient, and/or diluted by an excipient. When the excipient serves as a diluent, it can be a solid, semi-solid, or liquid material, which acts as a vehicle, carrier or medium for the active ingredient. For additional information regarding suitable methods and formulations for use in the present disclosure are found in REMINGTON'S PHARMACEUTICAL SCIENCES, Mace Publishing Company, Philadelphia, Pa., 17th ed. (1985).

According to one embodiment, the fullerenes may be administered alone, or in combination with any other medicament. Thus, the formulation may comprise fullerenes in combination with another active ingredient, such as a drug, in the same formulation. When administered in combination, the fullerenes may be administered in the same formulation as other compounds as shown, or in a separate formulation. When administered in combination, the fullerenes may be administered prior to, following, or concurrently with the other compounds and/or compositions.

In certain embodiments, the formulations comprise a skin-penetration enhancer. Any skin-penetration enhancer suitable for aiding the delivery of the fullerenes can be used. A list of skin-penetration enhancers can be found in, for example, Walters, K. A. and Hadgraft, J, Pharmaceutical Skin Penetration Enhancement, published by Marcel Dekker, New York, N.Y. (1993) and Osbourne, D. W., “Skin Penetration Enhancers cited in the Technical Literature,” Pharmaceutical Technology, pp 59-65 (November 1997).

The formulations can comprise from about 0.1% to about 99%, such as from about 0.1% to about 90%, about 5% to about 90%, or about 15% to about 75%, by weight of skin penetration enhancer. In certain embodiments the ratio of fullerenes to skin-penetration enhancer is from about 1:20 to about 1:10000, such as from about 1:60 to 1:300, on the basis of percentages by weight of total composition.

The fullerenes may be solubilized, especially when the fullerenes are hydrophobic. One method of solubilizing certain fullerenes is by formulation in liposomes. Other methods suitable for solubilizing certain fullerenes include the use of a solvent acceptable for use in the treatment of skin tissues and cells such as, but not limited to, DMSO (dimethylsulfoxide), alcohols, polyethylene glycol (PEG) wherein the size is less than PEG 1000 or any other solvent. Other solubilizers include glycol ethers, polyethylene glycol, polyethylene glycol derivatives, propylene glycol, propylene glycol derivatives, polysorbates (e.g., TWEEN™), fatty alcohols, aromatic alcohols, propylene glycol, glycerols, oils, surfactants, glucosides, and mixtures thereof. In certain embodiments the solubilizer is selected from diethylene glycol monoethyl ether (TRANSCUTOL®), polyethylene glycol of average molecular weight from 100 to 5000, triethylene glycol, tetraethylene glycol, pentaethylene glycol, hexaethylene glycol, septaethylene glycol, octaethylene glycol, propylene glycol, propylene glycol mono- and diesters of fats and fatty acids (e.g., propylene glycol monocaprylate, propylene glycol monolaurate), benzyl alcohol, glycerol, oleyl alcohol, mineral oil, lanolin/lanolin derivatives, petrolatum or other petroleum products suitable for application to the skin, propylene glycol mono- and diesters of fats and fatty acids, macrogols, macrogolglycerides or polyethylene glycol glycerides and fatty esters (e.g., stearoyl macrogolglycerides, oleoyl macrogolglycerides, lauroyl macrogolglycerides, linoleoyl macrogolglycerides), ethoxylated castor oil (e.g., Cremophor—a polyoxyl hydrogenated castor oil), C6-C30 triglycerides, natural oils, glucosides (e.g., cetearyl glucoside), surfactants, and mixtures thereof. In certain embodiments the formulations herein comprise from about 0.1% to about 99% by weight of solubilizer, such as from about 1% to about 75% by weight of solubilizer.

In certain embodiments the pharmaceutical compositions or formulations described herein have a viscosity at 20° C. of from about 5 cps to about 50000 cps, such as from about 500 cps to about 40000 cps, or about 5000 cps to about 30000 cps. Should the viscosity need to be adjusted it can be done by means of a viscosity modifying agent. Examples of viscosity modifiers include polyethylene glycols, acrylic acid-based polymers (carbopol polymers or carbomers), polymers of acrylic acid crosslinked with allyl sucrose or allylpentaerythritol (carbopol homopolymers), polymers of acrylic acid modified by long chain (C₁₀-C₃₀) alkyl acrylates and crosslinked with allylpentaerythritol (carbopol copolymers), poloxamers also known as pluronics (block polymers; e.g., Poloxamer 124, 188, 237, 338, 407), waxes (paraffin, glyceryl monostearate, diethylene glycol monostearate, propylene glycol monostearate, ethylene glycol monosterate, glycol stearate), hard fats (e.g., Saturated C₈-C₁₈ fatty acid glycerides), xantham gum, polyvinyl alcohol, solid alcohols, and mixtures thereof.

In certain embodiments the formulations contain one or more PEGs. Examples include at least one PEG of average molecular weight about 2000 or less, about 1500 or less, about 1000 or less, about 800 or less, about 600 or less, about 500 or less, or about 400 or less. Examples also include at least one PEG of average molecular weight about 3000 or more, about 3350 or more, or about 3500 or more. In one embodiment the formulation comprises a mixture of PEG's, such as at least one PEG having an average molecular weight of about 800 or less and at least one PEG having an average molecular weight of 3000 or more.

The formulation may comprise a variety of other components. Any suitable ingredient may be used herein but typically these optional component will render the formulations more cosmetically acceptable or provide additional usage benefits. Some examples of optional ingredients include, but are not limited to, emulsifiers, humectants, emollients, surfactants, oils, waxes, fatty alcohols, dispersants, skin-benefit agents, pH adjusters, dyes/colorants, analgesics, perfumes, preservatives, and mixtures thereof. In addition, the methods described herein include use of combination compositions comprising the fullerenes as described herein in combination with other agents suitable for treating pruritus.

Examples of suitable preservatives include but are not limited to parabens, benzyl alcohol, quaternium 15, imidazolidyl urea, disodium EDTA, methylisothiazoline, alcohols, and mixtures thereof. Examples of suitable emulsifiers include but are not limited to waxes, sorbitan esters, polysorbates, ethoxylated castor oil, ethoxylated fatty alcohols, macrogolglycerides or polyethylene glycol glycerides and fatty esters (e.g., stearoyl macrogolglycerides, oleoyl macrogolglycerides, lauroyl macrogolglycerides), esters of saturated fatty acids (e.g., diethylene glycol parmitostearate), macrogols of cetostearyl ether (e.g., macrogol-6-cetostearyl ether), polymers of high molecular weight, crosslinked acrylic acid-based polymers (carbopols or carbomers), and mixtures thereof. Examples of suitable emollients include but are not limited to propylene glycol dipelargonate, 2-octyldodecyl myristate, non-polar esters, triglycerides and esters (animal and vegetable oils), lanolin, lanolin derivatives, cholesterol, glucosides (e.g., cetearyl glucoside), pegylated lanolin, ethoxylated glycerides, and mixtures thereof. Examples of suitable surfactants include but are not limited to sorbitan esters, polysorbates, sarcosinates, taurate, ethoxylated castor oil, ethoxylated fatty alcohols, ethoxylated glycerides, caprylocaproyl macrogol-8 glycerides, polyglyceryl-6 dioleate, and mixtures thereof. Examples of suitable oils include but are not limited to propylene glycol monocaprylate, medium chain triglycerides (MCT), 2-octyl-dodecyl myristate, cetearyl ethylhexanoate, and mixtures thereof. Examples of suitable fatty alcohols include but are not limited to cetostearyl alcohol, cetyl alcohol, stearyl alcohol, and mixtures thereof. Also useful in the formulations herein are lipids and triglycerides (e.g., concentrates of Seed Oil Lipids, Concentrates of Marine Oil Lipids, high purity triglycerides and esters), alkyl ether sulfates, alkyl polyglycosides, alkylsulfates, amphoterics cream bases, and mixtures thereof.

Preparation of dry formulations that are reconstituted immediately before use also is contemplated. The preparation of dry or lyophilized formulations can be effected in a known manner, conveniently from the solutions of the invention. The dry formulations of this invention are also storable. By conventional techniques, a solution can be evaporated to dryness under mild conditions, especially after the addition of solvents for azeotropic removal of water, typically a mixture of toluene and ethanol. The residue is thereafter conveniently dried, e.g., for some hours in a drying oven.

The method herein is targeted to a widespread or localized area on the body of a subject having pruritus. The fullerene-containing preparations described above may be administered systemically or locally and may be used alone or as components of mixtures. In one embodiment the administration is local. The route of administration for the fullerenes may be topical, intradermal, intravenous, oral, or by use of an implant. In one embodiment the route of administration is topical. For example, fullerenes may be administered by means including, but not limited to, topical lotions, topical creams, topical pastes, topical suspensions, intravenous injection or infusion, oral intake, or local administration in the form of intradermal injection or an implant. Additional routes of administration are subcutaneous, intramuscular, or intraperitoneal injections of the fullerenes in conventional or convenient forms.

For topical formulations (such as ointments) to be applied to the surface of the skin, the concentration of the fullerenes in the excipient preferably ranges from about 0.001 to about 10% w/w, such as from about 0.005 to about 5% w/w, or from about 0.01 to about 1% w/w. The foregoing ranges are merely suggestive in that the number of variables with regard to an individual treatment regime is large and considerable deviation from these values may be expected.

When administered topically, the area to be treated may be massaged after application of the fullerenes.

Suitable isotonising agents are for example nonionic isotonising agents such as urea, glycerol, sorbitol, mannitol, aminoethanol or propylene glycol as well as ionic isotonising agents such as sodium chloride. Solutions containing fullerenes will contain the isotonising agent, if present, in an amount sufficient to bring about the formation of an approximately isotonic solution. The expression “an approximately isotonic solution” will be taken to mean in this context a solution that has an osmolarity of about 300 milliosmol (mOsm), conveniently 300+10% mOsm. It should be borne in mind that all components of the solution contribute to the osmolarity. The nonionic isotonising agent, if present, is added in customary amounts, i.e., preferably in amounts of about 1 to about 3.5 percent by weight, such as in amounts of about 1.5 to 3 percent by weight.

In one embodiment, the fullerenes are delivered topically. For topical administration, the fullerenes may be in standard topical formulations and compositions including lotions, creams, suspensions, serums, or pastes. Solubilized fullerenes can also be added to other dermatological products, such as hair gels, shampoos, conditioners, styling products, soaps, or the like. Injection may also be used when desired. Oral administration of suitable formulations may also be appropriate in those instances where the fullerenes may be readily administered to the widespread or localized area(s) on the body of the subject having pruritus via this route.

Generally, the pharmaceutical compositions or formulations described herein can be administered as a pharmaceutical, cosmetic, or nutritional formulation. These compositions or formulations can be administered topically, orally, intravenously, or as a suppository.

The present disclosure relates to use of any one or more of the fullerenes described herein for the treatment of pruritus. The present disclosure also relates to the manufacture of a medicament, particularly to the manufacture of a medicament for treating pruritus.

### Examples

### Example 1

In mice and humans substance P elicits scratching behavior at a central control point common to all itch pruritogens. It was hypothesized that the itch responses could be inhibited by water soluble fullerenes. To test this hypothesis an itch response was induced in mice using substance P. This is a common pre-clinical model used to test the efficacy of anti-itch compounds.

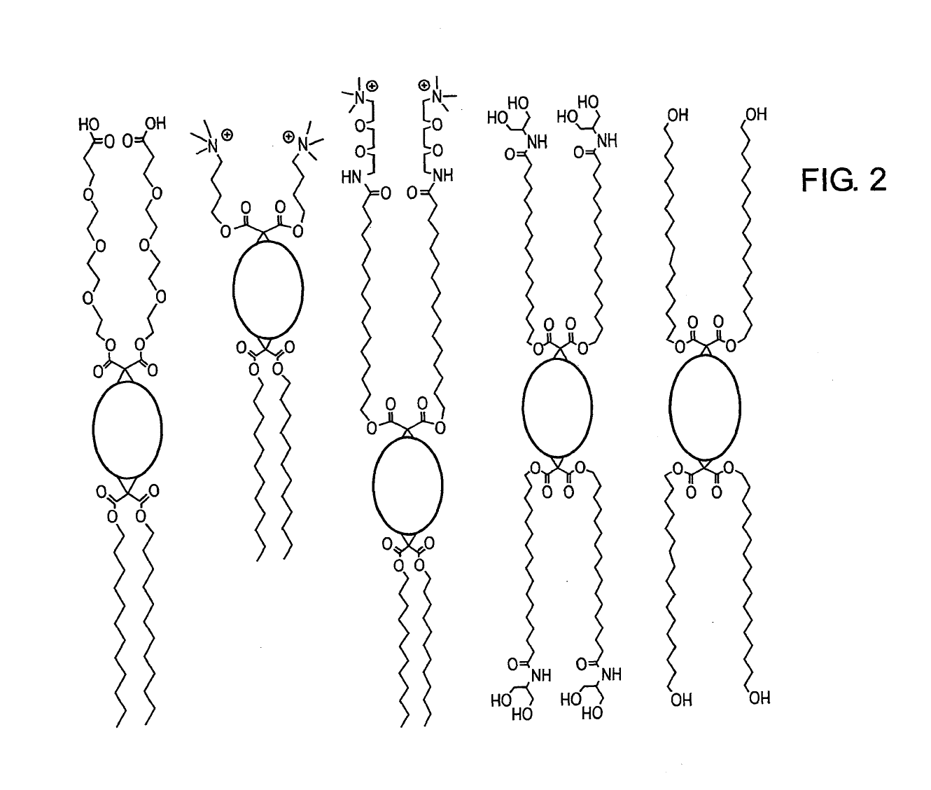

Balb/c mice (7-9 weeks) were individually housed in a plastic cage for at least 1 h before the experiment for acclimation. Compound 5 (ALM) (illustrated in FIGS. 1-2) or controls were injected intradermally in the interscapular part of the back (shaved 1 day prior to the experiment) 1 hour prior to injection with Substance P (300 ug/30 μl) in PBS. After injection, mice were returned to the cage, and their scratching behaviors were observed by an observer blinded to the contents of the injections.

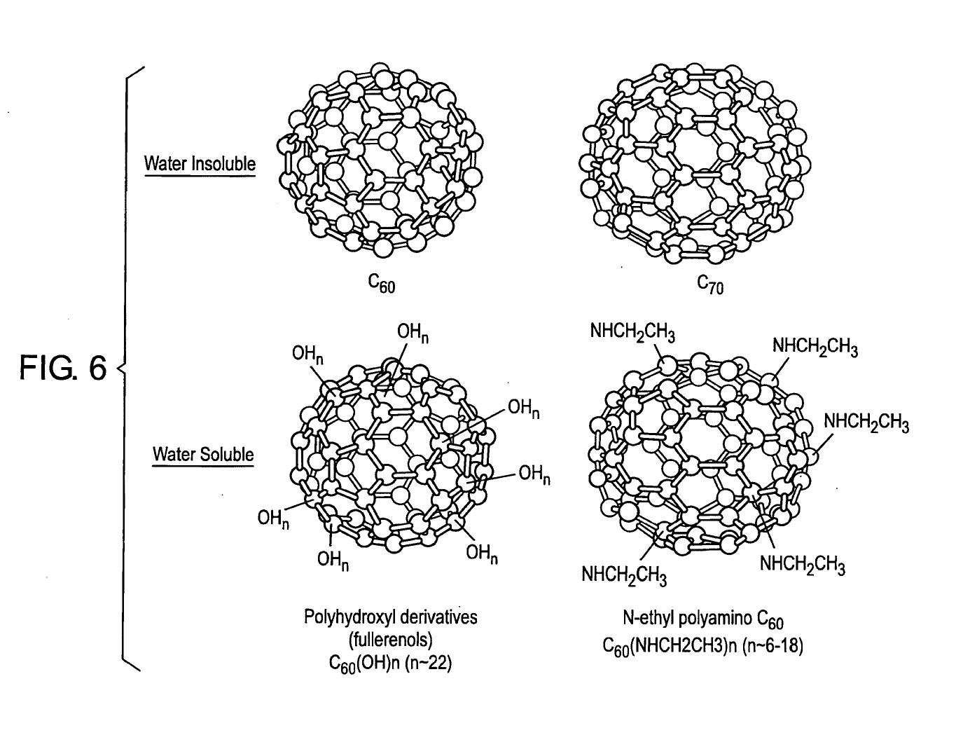

As seen in FIG. 6 the itch response in mice was significantly inhibited when compound 5 (ALM) was injected prior to substance P. Vehicle-treated animals scratched a mean of per 116±75.21 times per 30 min while animals treated with compound 5 (ALM) thirty minutes prior to challenge scratched a mean of 11.25±11.17 times per thirty minutes representing a statistically significant reduction (p⁼0.027) in scratching.

While various embodiments have been particularly shown and described herein, it will be understood by those skilled in the art that various changes in form and details may be made therein without departing from the spirit and scope of these embodiments as further defined by the appended claims.

1. A method of treating pruritus, comprising:
administering a therapeutically effective amount of one or more fullerenes to a subject in need thereof.

2. The method of claim 1, wherein said pruritus is caused by acute kidney failure, allergies, anaphylaxis, arthritis, athlete's foot, autoimmune hepatitis, blepharitis, candidiasis, cercarial dermatitis, chickenpox, chilblain, cholangitis, cholecystitis, chronic kidney failure, ciguatera poisoning, cirrhosis of the liver, cutaneous mastocytosis, decompression sickness, dermatitis, dermatitis herpetiformis, diabetes, drug allergies, dry skin, eczema, food allergies, heat rash, Hodgkin's disease, hyper-IgE syndrome, hyperthyroidism, hypothyroidism, jaundice, lichen sclerosis, lymphoma, mastocytosis, molluscum contagiosum, mycosis fungoides, non-Hodgkin's lymphoma, osteoarthritis, pancreatic cancer, pediculosis, pityriasis rosea, polycythemia, porphyria, primary biliary cirrhosis, primary sclerosing cholangitis, psoriasis, rabies, scabies, schistosomiasis, Sjogren's syndrome, slap-cheek syndrome, type 2 diabetes, Wiskott-Aldrich Syndrome, and/or yaws.

3. The method of claim 1, wherein said pruritus is induced by pruritogens.

4. The method of claim 3, wherein said pruritogens comprise urushiol.

5. The method of claim 1, wherein said pruritus is (i) immunologically-mediated, (ii) histamine-induced, (iii) non immunologically-mediated, (iv) parasite-induced, (v) fungus and bacteria induced, (vi) induced by insect bites, (vii) induced by plant derived pruritogens, and/or (viii) induced by poison ivy.

6. The method of claim 1, wherein said pruritus is widespread or localized.

7. The method of claim 1, wherein said fullerenes are administered as a pharmaceutical formulation.

8. The method of claim 7, wherein said pharmaceutical formulation is administered topically, orally, intravenously, or as a suppository.

9. The method of claim 7, wherein said pharmaceutical formulation further comprises at least one carrier and/or at least one excipient.

10. The method of claim 1, wherein said fullerenes are administered to the subject in combination with at least one other active ingredient.

11. The method of claim 1, wherein said pruritus is caused and/or aggravated by dry skin, allergic reactions, allergies, insect bites and stings, insect allergies, tick bites, flea bite, worm allergies, irritating chemicals, parasites, pregnancy, rashes, reactions to medicines, rash, itchy rash, skin inflammation, blisters, hives, eczema, contact dermatitis, poison oak, poison ivy, shingles, fungal and/or bacterial infection, lichen simplex, pityriasis, rosea, lichen sclerosis et atrophicus, nodular prurigo, vulval itch, chicken pox, measles, itch, anal itch, genital itch, reaction to medication, food allergy, jaundice, leukemia, polycythemia, kidney disease, hypothyroidism, hyperthyroidism, senile pruritus, fibers, psoriasis, eczema, dermatitis, sun burn, jaundice, liver diseases, uremia, polycythemia vera, lymphoma, Hodgkin's lymphoma, leukemia, jock itch, feminine itch, and/or psychogenic itch.

12. The method of claim 11, wherein said parasites comprise pinworms, scabies, lice, or a combination thereof.

13. The method of claim 11, wherein said dermatitis comprises atopic dermatitis, dermatitis herpetiformis, and solar dermatitis.

14. The method of claim 1, wherein said subject is a human.

15. The method of claim 1, wherein at least one of said one or more fullerenes is a synthetically modified fullerene of the formula
Zₘ—F—Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms.

16. The method of claim 15, wherein p is 60 or 70.

17. The method of claim 16, wherein p is 70.

18. The method of claim 1, wherein at least one of said one or more fullerenes is a synthetically modified fullerene of the formula
Z(Cₚ)Y
wherein p=60-200 carbons, preferably p=60 or 70; Y is a lipophilic moiety covalently connected to Cₚ, optionally through a linking group, at or near a pole thereof, and wherein Z is a lipophilic moiety, amphiphilic moiety, or a hydrophilic moiety covalently connected to Cₚ, optionally through a linking group, at or near a pole opposite to said Y.

19. The method of claim 18, wherein Cₚ is C₇₀.

20. The method of claim 19, wherein Z is a hydrophilic moiety.

21. The method of claim 1, wherein at least one of said one or more fullerenes is a synthetically modified fullerene of the formula
Z′ₘ—F—Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms.

22. The method of claim 21, wherein p is 60 or 70.

23. The method of claim 22, wherein p is 70.

24. The method of claim 1, wherein at least one of said one or more fullerenes is a synthetically modified fullerene of the formula
Z′(Cₚ)Y′
wherein: p=60-200 carbons, preferably p=60 or 70; Y′ is a hydrophilic or amphiphilic moiety covalently connected to Cₚ, optionally through a linking group, at or near a pole thereof, and wherein Z′ is a hydrophilic or amphiphilic moiety covalently connected to Cₚ, optionally through a linking group, at or near a pole opposite to said Y′.

25. The method of claim 24, wherein Cₚ=C₇₀.

26. The method of claim 1, wherein at least one of said one or more fullerenes is a compound shown in the present figures.

27. The method of claim 1, wherein at least one of said one or more fullerenes is selected from the group consisting of compounds 5, 7, 10, and 12.

28. A synthetically modified fullerene of the formula
Zₘ—F—Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms,
for use in treating pruritus.

29. A synthetically modified fullerene of the formula
Zₘ—F—Yₙ
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage.
Z and Y are positioned near respective opposite poles of Cₚ;
m=1-5 and Z is a hydrophilic, lipophilic, or amphiphilic chemical moiety;
n=1-5 and Y is a lipophilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms,
for preparation of a medicament for treating pruritus.

30. A synthetically modified fullerene of the formula
Z′hd m—F—Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms,
for use in treating pruritus.

31. A synthetically modified fullerene of the formula
Z′ₘ—F—Y′ₙ;
wherein F is a fullerene of formula Cₚ or X@Cₚ, the fullerene having two opposing poles and an equatorial region;
Cₚ represents a fullerene cage having p carbon atoms, and X@Cₚ represents such a fullerene cage having a chemical group X within the cage;
Z′ and Y′ are positioned near respective opposite poles of Cₚ;
m=1-5 and Z′ is a hydrophilic, lipophilic, or amphiphilic chemical moiety;
n=1-5 and Y′ is a hydrophilic or amphiphilic chemical moiety;
p=60-200 and p is an even number; and
X, if present, represents one or more metal atoms within the fullerene (F), optionally in the form of a trinitride of formula Gᵢ₌₁₋₃Hₖ₌₃₋ᵢN in which G and H are metal atoms,
for preparation of a medicament for treating pruritus.
